(19) United States
(12) Patent Application Publication
(10) Pub. No.: US 2009/0054874 A1
(43) Pub. Date: Feb. 26, 2009

(54) MULTI-LUMEN CATHETER INCLUDING A LUMEN HAVING A VARIABLE CROSS SECTIONAL AREA

(75) Inventors: William R. Barron, Riverton, UT (US); Murtaza Amin, Farmington, UT (US); Kelly B. Powers, North Salt Lake, UT (US)

Correspondence Address: Rutan & Tucker, LLP., 611 ANTON BLVD, SUITE 1400, COSTA MESA, CA, 92626, US

(73) Assignee: C. R. Bard, Inc., Murray Hill, NJ (US)

(21) Appl. No.: 12/197,919

(22) Filed: Aug. 25, 2008

Related U.S. Application Data
(60) Provisional application No. 60/957,636, filed on Aug. 23, 2007.

(51) Int. Cl. A61M 25/00

(52) U.S. Cl. 604/524

(57) ABSTRACT

A multi-lumen catheter configured for insertion into the vasculature of a patient for fluid infusion into or fluid aspiration from the patient is disclosed. The multi-lumen catheter includes one or more cross sectionally variable lumens, wherein the cross sectional area of the lumen(s) may be selectively increased, particularly during fluid infusion, in order to enable relatively greater fluid flow rate therethrough. In one embodiment, the multi-lumen catheter includes a deformable first septum for providing an increased cross sectional area for a lumen under high flow rate pressurization, such as power injection. A deformable second septum also deforms to allow for first septum deformation and additionally provides an urging force to restore the first septum to an un-deformed state once lumen pressurization has ceased. In another embodiment, a bi-positional septum is used to selectively increase the cross sectional area of a lumen of the catheter during power injection.

### CROSS-REFERENCE TO RELATED APPLICATIONS

This application claims the benefit of the U.S. Provisional Patent Application No. 60/957,636, filed Aug. 23, 2007, and entitled “MULTI-LUMEN POWER INJECTABLE CATHETERS AND METHODS OF USE,” which is incorporated herein by reference in its entirety.

### BRIEF SUMMARY

The present invention has been developed in response to the above and other needs in the art. Briefly summarized, embodiments of the present invention are directed to a multi-lumen catheter configured for insertion into the vasculature of a patient for fluid infusion into or fluid aspiration from the patient. The multi-lumen catheter includes one or more cross sectionally variable lumens, wherein the cross sectional area of the lumen(s) may be selectively increased, particularly during fluid infusion, in order to enable relatively greater fluid flow rate therethrough.

In one embodiment, the multi-lumen catheter includes a deformable first septum for providing an increased cross sectional area for a lumen under high flow rate pressurization, such as power injection. A deformable second septum, separating second and third lumens of the catheter, also deforms to allow for first septum deformation and additionally provides an urging force to restore the first septum to an un-deformed state once lumen pressurization has ceased.

In another embodiment, a bi-positional septum is used to selectively increase the cross sectional area of one of the lumens of the catheter during power injection, for example. When a respective one of the lumens is pressurized, the bi-positional septum is urged by the pressurization to move from a first position, wherein the lumen has a relatively small cross sectional area, to a second position having a relatively larger cross sectional area. Such increase in luminal cross sectional area enables power injection and other high fluid flow rate procedures to be carried out without having to replace the catheter with a larger size or fewer-numbered lumen catheter.

These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof that are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view of a catheter configured in accordance with one example embodiment of the present invention;

FIG. 2 is a cross sectional view of a catheter such as that shown in FIG. 1, showing a lumen configuration in accordance with one embodiment;

FIG. 3A is a cross sectional view showing the lumen configuration of FIG. 2 during pressurization of one of the lumens;

FIG. 3B is another cross sectional view showing the lumen configuration of FIG. 2 during pressurization of one of the lumens;

FIG. 4 is a cross sectional view of a catheter such as that shown in FIG. 1, showing a lumen configuration in accordance with another example embodiment;

FIG. 5 is a cross sectional view of a catheter such as that shown in FIG. 1, showing a lumen configuration in accordance with another example embodiment;

FIG. 6 is a cross sectional view of a catheter such as that shown in FIG. 1, showing a lumen configuration in accordance with another example embodiment;

FIG. 7 is a cross sectional view of a catheter such as that shown in FIG. 1, showing a lumen configuration in accordance with another example embodiment;

FIG. 8 is a cross sectional view of a catheter such as that shown in FIG. 1, showing a lumen configuration in accordance with another example embodiment;

FIG. 9A is a cross sectional view of a catheter such as that shown in FIG. 1, showing a lumen and bi-positional septum configuration in accordance with one example embodiment;

FIG. 9B is a cross sectional view showing the lumen and bi-positional septum configuration of FIG. 9A with the septum in a second position;

FIG. 10A is a cross sectional view of a catheter such as that shown in FIG. 1, showing a lumen and bi-positional septum configuration in accordance with another embodiment;

FIG. 10B is a cross sectional view showing the lumen and bi-positional septum configuration of FIG. 10A with the septum in a second position;

FIG. 11 is a cross sectional view of a catheter such as that shown in FIG. 1, showing a plurality of lumens and bi-positional septa in accordance with one example embodiment; and

FIG. 12 is a cross sectional view of a catheter such as that shown in FIG. 1, showing a plurality of lumens and bi-positional septa in accordance with one example embodiment of the present invention.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the invention, and are not limiting of the present invention nor are they necessarily drawn to scale.

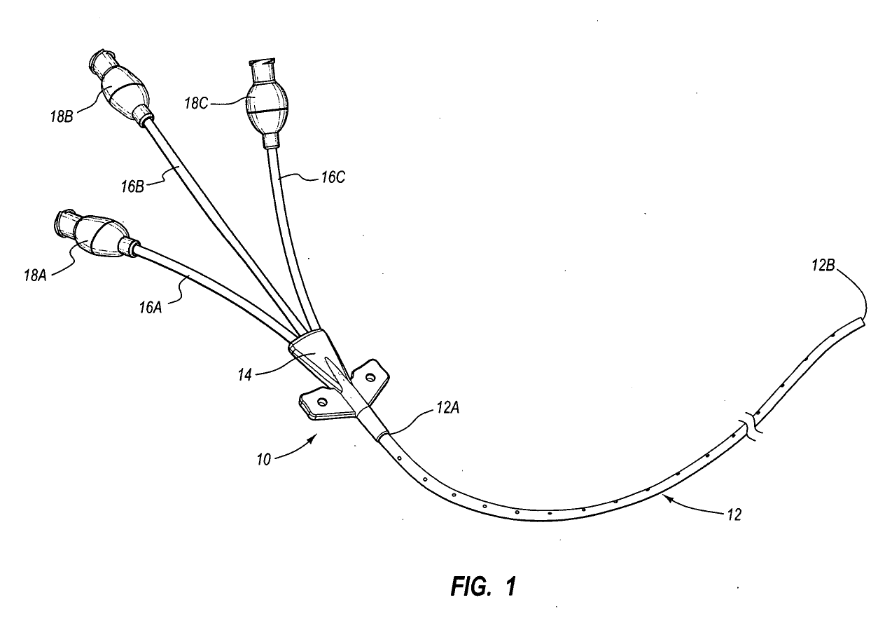

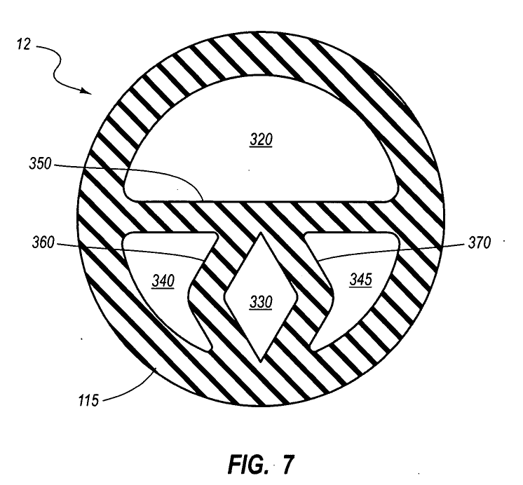

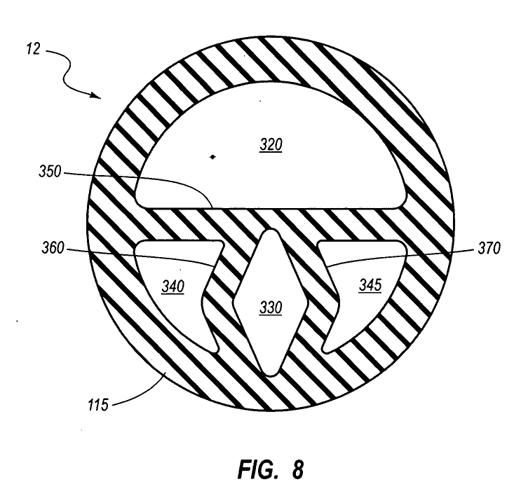

FIGS. 1-12 depict various features of embodiments of the present invention, which are generally directed to a multi-lumen catheter configured for insertion into the vasculature of a patient for fluid infusion into or fluid aspiration from the patient. The catheter to be described herein includes one or more cross sectionally variable lumens, wherein the cross sectional area of the lumens may be selectively increased when fluid pressure is applied, particularly during fluid infusion, in order to enable relatively greater fluid flow rate therethrough. As such, the cross sectionally variable lumen(s) are compliant and scalable in response to the application of pressure thereto.

Such selective luminal area increase is especially valuable in power injection scenarios, where high lumen flow rates are desirable in order to rapidly infuse contrast media or other fluids into the patient vasculature or other body portion. Some medical procedures, such as computed tomography (“CT”) scans, often require the relatively rapid infusion of contrast media fluid into a patient's vascular system. During such procedures, a proximal end of the inserted catheter assembly to be described is connected to a power injection machine. The injection pressure of the machine is set to a predetermined limit. When activated, the machine rapidly injects the media into the vasculature of the patient via the catheter assembly at a flow rate that will not exceed the predetermined fluid pressure limit. Fluids can be power injected into patients at flow rates ranging from about 2 cubic centimeters per second to greater than about 7 cubic centimeters per second. The selective and reversible (recoverable) increase in the lumen cross sectional area in the present multi-lumen catheter to be described herein enables power injection through the selected lumen without increasing the overall size of the catheter or compromising use and patency of the remaining catheter lumens during nominal flow rate infusion or aspiration procedures.

Reference is first made to FIG. 1, which depicts a catheter, generally designated at 10 and configured in accordance with one example embodiment of the present invention. As shown, the catheter 10 includes a body 12 having a proximal end 12A, a distal end 12B, and defining multiple lumens extending therebetween. In the present embodiment, the catheter is a peripherally inserted central catheter (“PICC”), though in other embodiments other types of catheters having a variety of size, lumen, and prescribed use configurations can benefit from the principles described herein. Further, though shown here with an open distal end, the catheter in other embodiments can have a closed or valved distal end. As such, the present discussion is presented by way of example and should therefore not be construed as being limiting of the present invention in any way.

A hub 14 is included at the catheter proximal end 12A. The hub 14 permits fluid communication between extension tubing 16A, 16B, 16C and the lumens of the catheter body 12. Each extension tubing component 16A-16C respectively includes on a proximal end thereof a connector 18A, 18B, 18C for enabling the catheter 10 to be operably connected to one or more of a variety of medical devices, including syringes, pumps, infusion sets, etc. Again note that the particular design and configuration of the afore-described components is exemplary only.

A distal portion of the catheter body 12 is configured for insertion within the vasculature of a patient. So positioned, the catheter 10 is utilized to infuse fluids into the patient vasculature, or to aspirate fluids therefrom. In one application, contrast media or other fluid is power injected, or infused into the patient vasculature at a relatively high fluid flow rate, typically from about 2 to greater than about 7 cubic centimeters (“cc”) per second, so as to enable improved imaging during a computed tomography (“CT”) scan of the patient body. Examples of catheters designed to accommodate the relatively high pressures resulting from power injection of fluids into the patient vasculature are described in U.S. Patent Publication Nos. 2004/0243103 and 2006/0149214, each of which is incorporated herein by reference in its entirety. Note that in other embodiments, the catheter can be configured to infuse or aspirate fluids from a portion of the patient's body other than the vasculature.

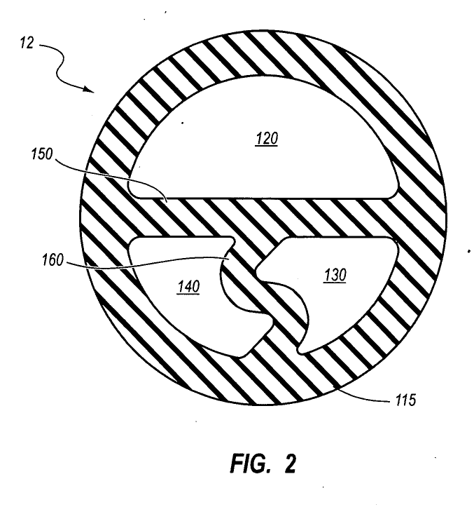

Reference is now made to FIG. 2 in describing features of the catheter 10, according to one embodiment. As shown, the catheter body 12 is defined by a wall 115 and further includes a first lumen 120, a second lumen 130, and a third lumen 140 extending from the proximal end 12A to the distal end 12B of the body. The first lumen 120 is configured in the present embodiment to withstand pressures associated with power injection of fluids, such as contrast media, therethrough. As such, the first lumen 120 can accommodate fluid flow rates ranging from about 2 cc/sec. to greater than about 7 cc/sec. In the present embodiment, the second lumen 130 and third lumen 140 define substantially equal cross-sectional areas, though in other embodiments the relative cross sectional areas of the three lumens may vary from what is shown and described.

The first lumen 120 is separated from the second lumen 130 and the third lumen 140 lumens by a first septum 150 extending longitudinally along the length of the catheter body 12 and radially across the cross sectional width of the catheter body. The second lumen 130 and third lumen 140 are separated from one another by a second septum 160 that also longitudinally extends along the length of the catheter body 12 and radially extending from the catheter body wall 115 to the first septum 150. Note that the contact point of the second septum 160 with the first septum 150 is at a midpoint of the first septum, but that the contact point could be in other locations along the first septum in other embodiments.

The second septum 160 is configured in the present embodiment to be resiliently deformable such that it can be deformed when subjected to sufficient force via the first lumen 120, but restored to its un-deformed shape (as shown in FIG. 2) when the force is removed. As seen in FIG. 2, the second septum is S-shaped to facilitate such resilient deformation. Note, however, that other shapes and septum configurations can also be employed to perform the intended function.

Likewise, the first septum 150 is also resiliently deformable so as to enable it to deform when subjected to a sufficient force, such as when the first lumen 120 is pressurized by power injecting contrast media or other fluid therethrough at a relatively high fluid flow rate.

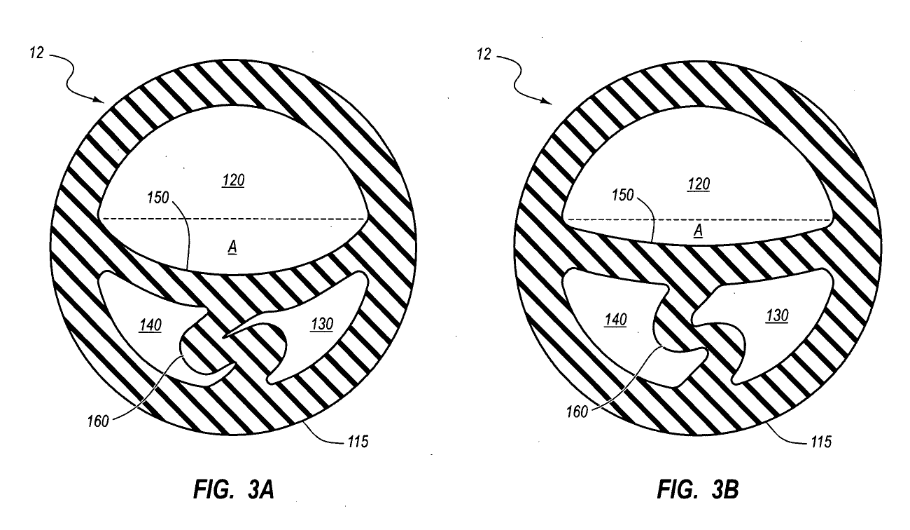

FIGS. 3A and 3B show the changes to the lumen arrangement of the catheter body 12 when the first lumen 120 is pressurized. As can be seen, pressurization of the first lumen 120 causes the first septum 150 to deform, thereby expanding the cross sectional area of the first lumen 120 by an additional areal amount A, seen in FIG. 3A. This enables the first lumen 120 to provide adequate volume for power injection of contrast media or other fluid. In one possible implementation, the first lumen 120 increases in cross-sectional area up to approximately 100% of its original cross-sectional area during lumen pressurization such as, for example, in the case of power injection.

FIG. 3B shows that as the fluid pressure present in the first lumen 120 decreases, either by reduction of fluid flow into the catheter 10 or by fluid pressure attenuation in more distal portions of the catheter body 12, deformation of the first septum 150—and hence size of the additional area A—decreases in magnitude. Generally, pressure will be relatively greater in more proximal portions of the first lumen 120, and relatively less in more distal portions during power injection or other lumen pressurization. The S-shape of the second septum 160 is shown as substantially compressed in FIG. 3A when the first lumen 120 is under a net pressurization. The second septum 160 is compressed in one embodiment until the mechanical strength of the second septum in its compressed or deformed state equalizes with the deformation force imparted to it via pressurization of the first lumen 120. The second septum 160 is relatively less compressed in FIG. 3B when the first lumen net pressurization is reduced, and substantially uncompressed in FIG. 2 when no net pressurization is present.

Due to its S-shaped configuration, the second septum 160 provides an urging force to restore the first septum 150 to restore its un-deformed shape, shown in FIG. 2, when the net pressurization of the first lumen 120 is removed. As such, the second septum 160 serves as one example of a septum assembly that facilitates resilient deformation of the first septum 150 while also facilitating elastic restoration, i.e., mechanical recovery, of the un-deformed shape of the first septum when the first lumen 120 is unpressurized. In some embodiments the septum assembly provides an urging force to return the first septum to its un-deformed state, while in other embodiments the septum assembly merely provides a counteracting force in limiting deformation of the first septum under pressurization. In either case, the septum assembly facilitates restoration of the first septum to its un-deformed state either actively, by providing an urging force to the first septum, or passively by not inhibiting the first septum to return to its un-deformed state.

It is appreciated that the magnitude of septum deformation under an applied fluid pressure for both the first and second septa 150, 160 is determined by the geometry of each septum as well as the corresponding structural strength of the septa. Generally, therefore, septum deformation is most pronounced, for example, where the septum wall thickness is relatively thin and where the septum is unsupported for an extended radial distance.

The deformable septa 150, 160 of the catheter 10 as depicted and described in connection with FIGS. 2-3B provide the catheter with a lumen, i.e., the first lumen 120, having a variable cross sectional area. As such, the first lumen 120 can serve as a lumen with a nominal cross sectional area during normal infusion/aspiration applications, but also serve as an expanded area power injectable lumen when high fluid flow rates through the lumen are needed. Once the need for high fluid flow is no longer needed and the applied pressure is removed, the first lumen 120 can recover to its substantially un-deformed, nominal state as shown in FIG. 2 with the assistance of the mechanically restorative force provided by the septum assembly.

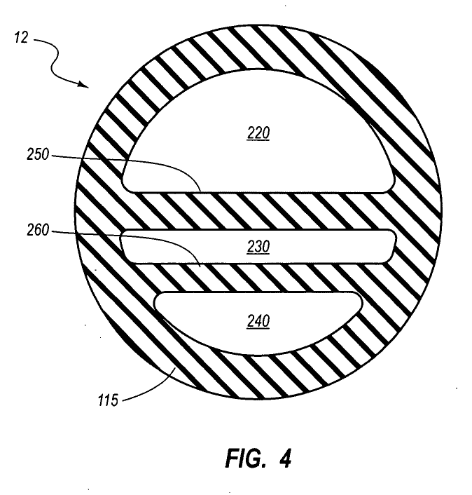

Note that various other possible septum configurations can achieve the intended function as described above. FIGS. 4-8 show several such exemplary configurations. As many aspects of the catheter configurations shown in these figures are similar to those already described in connection with FIGS. 2-3B, only selected aspects are discussed in detail below. In FIG. 4, the catheter body 12 includes a first lumen 220, second lumen 230, and third lumen 240 disposed in a stacked arrangement within the catheter body. The first lumen 220 is configured to accommodate power injectable fluid flow rates, typically ranging from about 2 to greater than about 7 cc/sec. The first lumen 220 is separated from the second lumen 230 by a first septum 250, while the second lumen 230 is separated from the third lumen 240 by a second septum 260, which is disposed radially parallel to the first septum.

When the first lumen 220 is pressurized, as in a power injection procedure, deformation of the first septum 250 occurs in a manner similar to that described in connection with FIGS. 2-3B. Deformation forces are distributed along the first septum 250 and are countered by the second septum 260, which also deforms as a result of the deformation forces acting upon the first septum. When net pressurization of the first lumen 220 is removed, the second septum substantially returns to its un-deformed configurations and urges the first septum 250 to substantially return to its un-deformed configuration. Thus, the second septum 260 serves as another example of a septum assembly that facilitates resilient deformation of the first septum 250 while also facilitating restoration of the un-deformed shape of the first septum when the first lumen 220 is no longer pressurized.

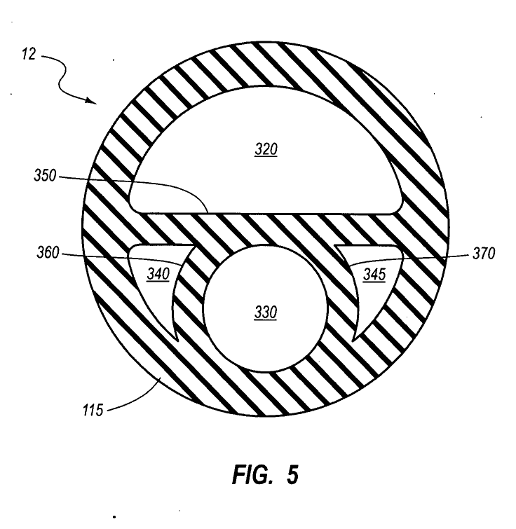

FIGS. 5-8 depict further possible septum assembly configurations: FIG. 5 shows a quad lumen profile, including first, second third, and fourth lumens 320, 330, 340, and 345, respectively. A septum assembly including a second septum 360 and a third septum 370 divide the second, third, and fourth lumens 330, 340, 345. The second septum 360 and third septum 370 join with a first septum 350 and each resiliently deforms to enable the first lumen to deform when the first lumen 320 is pressurized, thereby increasing the relative cross sectional area of the first lumen as before. Once the first lumen 320 is no longer pressurized, the second and third septa 360, 370 urge the first septum 350 into its un-deformed configuration. Thus, the second septum 360 and third septum 370 together serve as another example of a septum assembly that facilitates resilient deformation of the first septum 350 and restoration of the un-deformed shape of the first septum when the first lumen 320 is no longer pressurized.

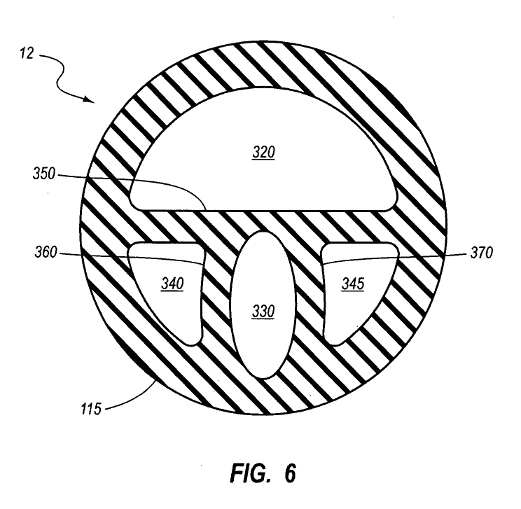

FIGS. 6-8 show variations of the embodiment of FIG. 5, wherein the second, third, and fourth lumens 330, 340, and 345 define various cross sectional shapes, including oval, triangle, and diamond. Thus, these and other possible configurations are contemplated as included within the claims of the present invention.

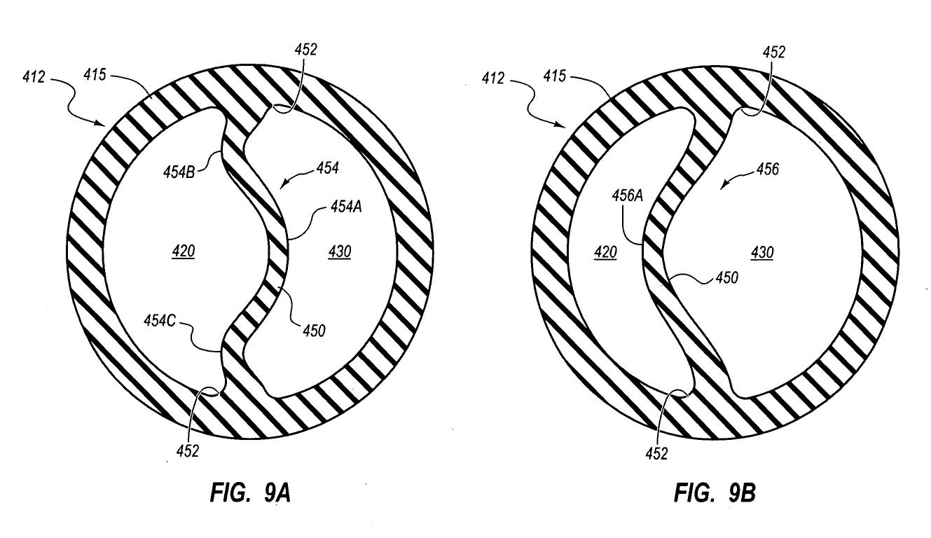

Reference is now made to FIGS. 9A and 9B, which depict a multi-lumen catheter including lumens having variable cross sectional areas, according to one example embodiment. As shown, the catheter includes a body 412 defined by a wall 415. The wall 415 further defines outer boundaries for a first lumen 420 and a second lumen 430, which lumens are separated by a flexible, bi-positional septum 450 that longitudinally extends the length of the catheter body 412. The septum 450 joins the body wall 415 at contact points 452.

As can be seen, the septum 450 has a radial width that is greater than the inner diameter of the wall 415 measured between the contact points 452. So configured, the septum 450 is positionable between a first position 454, shown in FIG. 9A, and a second position 456, shown in FIG. 9B. In the configuration of FIG. 9A, either of the first and second lumens 420 and 430 can be employed for nominal pressure fluid infusion/aspiration. Should power injection or other relatively high flow rate infusion be desired via the second lumen 430, for instance, the second lumen will be pressurized upon commencement of infusion. Upon pressurization, the septum 450 is moved by the pressure in the second lumen 430 from the first position 454 shown in FIG. 9A to the second position 456 shown in FIG. 9B. This movement of the septum 450 increases the cross sectional area of the second lumen 430, thus enabling a high flow rate infusion to pass therethrough. Note that the first lumen 420 remains usable for standard flow infusion/aspiration. Once net pressurization of the second lumen 430 is ceased, the septum 450 remains in the second position 456, thus enabling later nominal or high flow rate fluid infusion to occur via the second lumen. This aspect avoids potential problems with blood suck-up by the smaller area lumen when the enlarged lumen reduces in size after pressurization is removed.

Should high flow rate infusion be subsequently desired via the first lumen 420, however, the first lumen will be pressurized upon commencement of infusion. Upon pressurization, the septum 450 is moved by the pressure in the first lumen 420 from the second position 456 shown in FIG. 9B to the first position 454 shown in FIG. 9A. As was the case with the second lumen 430 previously, movement of the septum 450 to the first position 454 increases the cross sectional area of the first lumen 420, thus enabling a high flow rate infusion to pass therethrough. Again, once net pressurization of the first lumen 420 is ceased, the septum 450 remains in the first position 454, thus enabling later nominal or high flow rate fluid infusion to occur via the first lumen.

Though the septum 450 can be moved between the first position 454 and the second position 456 as just described, each of these positions is a position of stability or repose, e.g., a “local minimum energy” for the septum. In this way, stable and selectable bi-positioning of the septum 450 is possible.

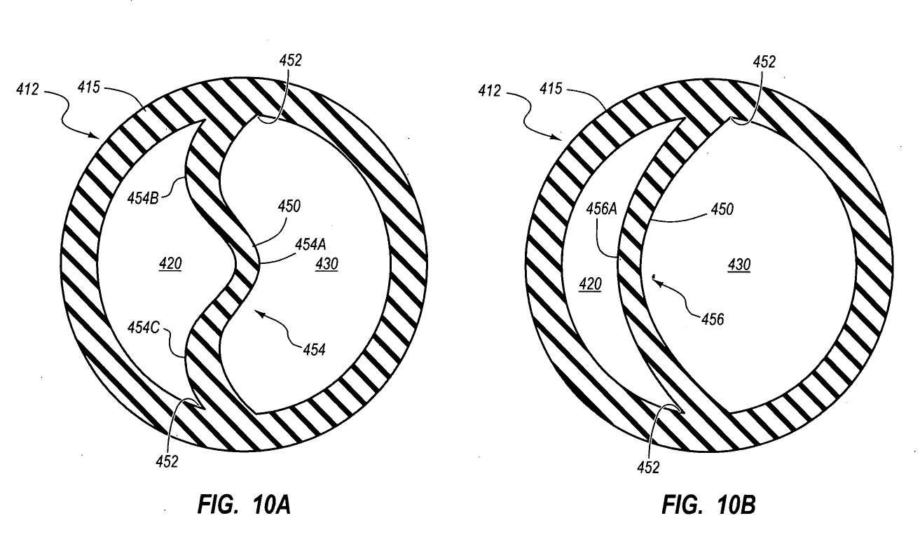

Various modifications to the principle of operation described and depicted in connection with FIGS. 9A and 9B can be employed. For example, FIGS. 10A and 10B show the septum 450 configured so as to create a relatively larger second lumen 430 when the second lumen is in a pressurized state, i.e., the septum in the second position 456.

Note further that in the configurations shown in FIGS. 9A and 10A, the septum 450 in the first position 454 defines a convexly shaped cross sectional curve that includes three nodes indicated at 454A, B, and C, respectively. In the second position 456 of FIGS. 10A and 10B, the septum 450 defines a concavely shaped cross sectional curve that includes only one node 456A. Of course, in other embodiments, more or fewer nodes may be included on the septum.

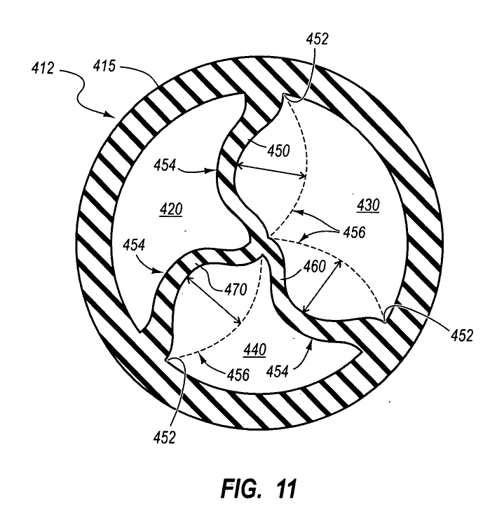

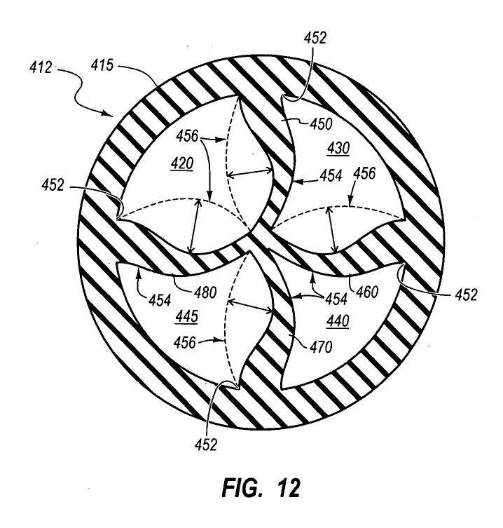

FIGS. 11 and 12 indicate that the principle described in connection FIGS. 9A-10B can be expanded so as to include three bi-positional septa 450, 460, 470 separating first, second, and third lumens 420, 430, and 440 as in FIG. 11, or four bi-positional septa 450, 460, 470, 480 separating first, second, third, and fourth lumens 420, 430, 440, and 445 as in FIG. 12. Thus, the principles described herein can be expanded to catheters having two, three, four, or more lumens, with one or more lumens being power injectable.

The catheters disclosed herein may be manufactured from any suitable material, including, without limitation, polymers, elastomers, thermoplastics, and, more specifically, polyurethane. The catheters disclosed herein may have any durometer ratings suitable for the described application, ranging, for example, from 60 Shore A to 70 Shore D.

The present invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope. The words “including,” “has,” and “having,” as used herein, including the claims, shall have the same meaning as the word “comprising.”

What is claimed is:

1. A multi-lumen catheter, comprising:
a body including an outer wall extending between a proximal end and a distal end thereof;
a first septum extending longitudinally within the body between the proximal and distal ends thereof, the first septum at least partially defining a first lumen having a first cross sectional area in an un-deformed configuration; and
a septum assembly extending longitudinally within the body between the proximal and distal ends thereof, the septum assembly at least partially defining a second lumen and a third lumen, wherein the septum assembly is deformable in response to deformation of the first septum when the first lumen is pressurized, the deformation of the first septum increasing the first cross sectional area of the first lumen to a second cross sectional area, and wherein the septum assembly provides an urging force to return first septum to an un-deformed configuration when the first lumen is no longer pressurized.

2. The multi-lumen catheter as defined in claim 1, wherein the septum assembly is attached to the first septum.

3. The multi-lumen catheter as defined in claim 1, wherein the septum assembly includes a mechanical strength that balances a force provided by the pressurized first lumen so as to inhibit further deformation of the first septum.

4. The multi-lumen catheter as defined in claim 1, wherein the catheter is a peripherally inserted central catheter.

5. The multi-lumen catheter as defined in claim 1, wherein the first lumen is capable of being pressurized for power injection by a fluid flow rate of from about 2 cc/second to greater than about 7 cc/second.

6. The multi-lumen catheter as defined in claim 1, wherein the septum assembly includes a second septum.

7. The multi-lumen catheter as defined in claim 6, wherein the second septum attaches to the first septum and has an S-shaped configuration.

8. The multi-lumen catheter as defined in claim 6, wherein the second septum extends parallel to the first septum.

9. The multi-lumen catheter as defined in claim 1, wherein deformation of the first septum reduces cross sectional areas in at least one of the second and third lumens from a first cross sectional area to a second cross sectional area.

10. The multi-lumen catheter as defined in claim 1, further comprising a fourth lumen at least partially defined by the septum assembly, wherein deformation of the first septum reduces a first cross sectional area of the fourth lumen from a first cross sectional area to a second cross sectional area.

11. The multi-lumen catheter as defined in claim 10, wherein at least one of the second, third, and fourth lumens has one of a round, oval, triangular, and/or rectangular cross sectional shape.

12. A method for pressurizing an internal first lumen of a catheter, the first lumen being at least partially defined by a first septum, the first lumen defining a first cross sectional area in an un-deformed configuration, the catheter further including an internal second lumen and an internal third lumen, the second and third lumens being separated by a septum assembly, the method comprising:
pressurizing the first lumen so as to cause deformation of the first septum from the un-deformed configuration to a deformed configuration and to cause the first lumen to define a second cross sectional area, deformation of the first septum further causing deformation of the septum assembly; and
depressurizing the first lumen so as to enable the septum assembly to urge the first septum to the un-deformed configuration.

13. The method for pressurizing as defined in claim 12, wherein pressurizing the first lumen further includes pressurizing the first lumen by a fluid flow rate of from about 2 cc/second to greater than about 7 cc/second.

14. The method for pressurizing as defined in claim 12, wherein deformation of the first lumen causes a reduction of cross sectional area of the second and third lumens separated by the septum assembly.

15. The method for pressurizing as defined in claim 12, wherein pressurizing the first lumen further comprises:
pressurizing the first lumen so as to cause deformation of the first septum until a mechanical strength of the septum assembly prevents further deformation of the first septum.

16. The method for pressurizing as defined in claim 12, wherein the septum assembly includes a second septum attached to the first septum and wherein pressurizing the first lumen further comprises pressurizing the first lumen so as to cause compression of the second septum, and wherein depressurizing the first lumen further comprises depressurizing the first lumen so as to enable decompression of the second septum.

17. The method for pressurizing as defined in claim 12, further comprising:
inserting the catheter into a vasculature of a patient before pressurizing the first lumen.

18. A multi-lumen catheter, comprising:
a catheter body extending between a proximal and a distal end; and
at least one bi-positional septum disposed within the catheter body, the at least one bi-positional septum at least partially separating an internal first lumen from an internal second lumen, wherein the at least one bi-positional septum is movable between a first position and a second position when a respective one of the first and second lumens is pressurized, and wherein the at least one bi-positional septum remains in the respective first or second position when the pressurization is removed from the respective first or second lumen.

19. The multi-lumen catheter as defined in claim 18, wherein the at least one bi-positional septum includes a convex cross sectional shape in the first position and a convex cross sectional shape in the second position.

20. The multi-lumen catheter as defined in claim 18, wherein the at least one bi-positional septum in the first position defines a cross sectional curved shape including three nodes, and wherein the at least one bi-positional septum in the second position defines a cross sectional curved shape including one node.

21. The multi-lumen catheter as defined in claim 18, wherein the catheter body includes three bi-positional septa that are joined together at a common contact point, the three bi-positional septa separating first, second, and third lumens.

22. The multi-lumen catheter as defined in claim 18, wherein the catheter body includes four bi-positional septa that are joined together at a common contact point, the four bi-positional septa separating first, second, third, and fourth lumens.

23. The multi-lumen catheter as defined in claim 18, wherein at least one of the first and second lumens is capable of being pressurized by a fluid flow rate of from about 2 cc/second to greater than about 7 cc/second.

24. The multi-lumen catheter as defined in claim 18, wherein the first position of the at least one bi-positional septum defines a first position of stability first local minimum energy and wherein the second position of the at least one bi-positional septum defines a position of second local minimum energy.

25. The multi-lumen catheter as defined in claim 18, wherein the catheter body includes only two lumens, and wherein the at least one bi-positional septum attaches to an outer wall of the catheter body at a first contact point and a second contact point, and wherein a width of the at least one bi-positional septum is relatively greater than a linear distance measured from the first contact point to the second contact point.

26. The multi-lumen catheter as defined in claim 18, wherein a width of the at least one bi-positional septum is relatively greater than an inner diameter of the catheter body.

27. A method for pressurizing a lumen of a multi-lumen catheter, the catheter including at least an internal first and an internal second lumen at least partially separated by at least one bi-positional septum, the method comprising:
pressurizing the first lumen so as to cause the at least one bi-positional septum to move from a first position to a second position and to cause the first lumen to expand from a first cross sectional area to a second cross sectional area; and
depressurizing the first lumen, the at least one bi-positional septum remaining in the second position when the first lumen is depressurized.

28. The method for pressurizing as defined in claim 27, wherein the pressurizing the first lumen further comprises:
pressurizing the first lumen so as to cause the at least one bi-positional septum to move from a first position of stability to a second position of stability.

29. The method for pressurizing as defined in claim 27, wherein pressurizing the first lumen so as to cause the at least one bi-positional septum to move from the first position wherein the at least one bi-positional septum includes a convex cross sectional shape to the second position wherein the at least one bi-positional septum includes a concave cross sectional shape.

30. The multi-lumen catheter as defined in claim 27, wherein at least one of the first and second lumens is capable of being pressurized for power injection by a fluid flow rate of from about 2 cc/second to greater than about 7 cc/second.
